(19) United States
(12) Patent Application Publication
(10) Pub. No.: US 2016/0120611 A1
(43) Pub. Date: May 5, 2016

(54) Medical Robot

(71) Applicant: KUKA LABORATORIES GMBH, Augsburg (DE)

(72) Inventor: Sebastian Lohmeier, Munchen (DE)

(21) Appl. No.: 14/783,284

(22) PCT Filed: Mar. 13, 2014
(86) PCT No.: PCT/EP2014/000680

§ 371 Date: Nov. 20, 2015

(30) Foreign Application Priority Data
Apr. 8, 2013 (DE) 10 2013 005 982.8

(51) Int. Cl. A61B 34/30; A61B 90/50

(52) U.S. Cl. CPC A61B 34/30; A61B 90/50; A61B 2090/5025; A61B 2017/00477

(57) ABSTRACT

A medical robot according to the invention comprises a base, an instrument flange for attaching a minimally invasive instrument, which instrument flange is connected to the base in such a way that the instrument flange can be moved by means of an actuated kinematic system, and a tube flange for attaching a tube, wherein the tube flange is connected to the kinematic system by means of a movable joint assembly, which has at least one actuated, in particular electrically actuated, and/or at least one elastically bound passive joint and/or at least one lockable joint.

The present invention relates to a medical robot for guiding a minimally-invasive instrument, as well as to a medical robot device with a suitable medical robot.

A medical robot for guiding a minimally-invasive instrument is known from EP 1 015 068 A1. The robot guides the instrument into a patient through a tube, which is rigidly connected to the kinematics of the robot.

The task of the present invention is to make available an improved medical robot, respectively an improved medical robot device.

This task is accomplished through the characteristics of the independent claims, which contain dependent claims regarding advantageous further developments.

A medical robot according to one embodiment of the present invention has a base. This can be inertial or fixed to its surroundings, in particular permanently or detachably connected with a floor, a wall or a ceiling of a stationary or mobile operating room. Likewise, it can be permanently or detachably connected with a stationary or mobile operating table. The base can also be located on a mobile platform, in particular an autonomous platform.

The medical robot has an instrument flange which is equipped for coupling a minimally-invasive instrument. A medical robot device according to one embodiment of the present invention can have one or more, in particular different, minimally invasive instruments, which—in particular alternately—can be coupled permanently or detachably to the instrument flange. The instrument flange can in particular have a signal and/or energy supply interface for signal and/or power connection to the instrument and/or a mechanical interface for permanent or detachable mounting of the instrument.

A minimally invasive instrument coupled or able to be coupled to the medical robot has in one embodiment an instrument shaft with an end effector, which is provided or adapted for insertion into a patient.

The end effector can in particular have, or in particular be, a scalpel, shears, forceps or a clamp, an optical output and/or input opening for sending and/or receiving electromagnetic radiation, in particular visible, and/or a fluid output and/or input opening for supplying and/or discharge of in particular liquid and/or gaseous fluid.

In order to insert the end effector or a portion of the instrument shaft into the patient, the medical robot device has, in one embodiment of the present invention, one or more tubes, in particular different tubes, in particular trocars or tubes with a tappet removably guided therein, with a sharp or blunt cutting edge.

The medical robot can, in one embodiment, be adapted to move the instrument shaft around a trocar point, in particular an invariant one or one fixed to its surroundings, which in one embodiment is located in the tube. In particular, the medical robot can be adapted, in particular in its kinematics, to move the minimally invasive instrument in translation in the direction of a longitudinal axis of the instrument which is aligned with an axial direction of the tube and/or can contain the trocar point, in particular in order to insert it (more deeply) into the patient or (at least in part) withdraw it from the patient. Additionally or alternatively, the medical robot can be adapted, in particular in its kinematics, to rotate the minimally invasive instrument about the longitudinal axis of the instrument. Additionally or alternatively, the medical robot can be adapted, in particular in its kinematics, to rotate the minimally invasive instrument about one or two axes of rotation, which in one embodiment are at least substantially perpendicular to one another and/or positioned on the longitudinal axis of the instrument and/or intersect at the trocar point. In this manner, the kinematics of the medical robot can constitute a translational and/or one, two or three rotational degrees of freedom of the end effector about the trocar point.

Additionally or alternatively, the end effector has in one embodiment one or more additional intra-corporal degrees of freedom, for example one, two or more rotational degrees of freedom about rotational axes spaced away from the trocar point. An intra-corporal degree of freedom is understood to mean primarily a degree of freedom or an ability to move the end effector relative to the instrument shaft.

One or more such intra-corporal degrees of freedom can be actuated extra-corporally in one embodiment. In particular, a single- or multi-axis drive can be positioned on the instrument shaft for driving the end effector. In one further development, the end effector is coupled with the extra-corporal drive mechanically, in particular through one or more tension and/or pressure means and/or rotation shafts, pneumatically, hydraulically and/or electrically.

The instrument flange is adjustably attached to the base using actuated kinematics of the medical robot. The kinematics has, in one embodiment, at least six, in particular at least seven joints. By means of kinematics with six joints, a three-dimensional position and a three-dimensional orientation of the instrument flange can be represented. In particular, the aforementioned degrees of freedom about the trocar point can be represented, or actuated, by six-axis kinematics. Kinematics with seven or more joints is redundant and advantageously makes possible, in one embodiment, the representation of the same three-dimensional position and orientation of the instrument flange with different postures or different joint positions, in particular so as to avoid interference between interacting medical robots.

In one embodiment, one or more, in particular all joints of the kinematics, are rotary joints, in particular with one rotary degree of freedom about a joint or axis of rotation. In a further development, two or more, in particular all consecutive rotary or joint axes of the kinematics are positioned at right angles to one another. An advantageous structure and dynamics are thereby provided. The joints are electrically actuated in one embodiment, in particular by electric motors.

According to one aspect of the present invention, the medical robot has a tube flange, in particular for detachably coupling a tube, the tube flange being connected through an additional movable articulated coupling with the kinematics. The tube flange in one embodiment is designed for frictional and/or positive torque-proof and/or axially fixed coupling of a tube.

Compared to a free or unconnected tube, a tube that is connected with the kinematics can, in one embodiment, reduce undesired relative motion of the minimally invasive instrument, and in particular support or embed the instrument shaft. Additionally or alternatively, operating precision can be increased, in particular through more exact positioning of the instrument and tube relative to one another. Additionally or alternatively, the burden on a patient during insertion and/or removal and/or movement of a minimally invasive instrument can be reduced. Additionally or alternatively, a tube which is connected with the kinematics can simplify, in one embodiment, the insertion and removal of an instrument and/or changing an instrument if the tube does not need to be manipulated by the user. Thus, in one embodiment, changing an instrument can advantageously be carried out one-handed.

A tube rigidly connected to the kinematics, as in the aforementioned EP 1 015 068 A1, allows a final joint axis of the kinematics, which is aligned with the longitudinal axis of the tube and is translational, to be fixed so as to mount the tube in a fixed manner to the patient or invariantly during insertion and removal of the instrument using kinematics. In addition, the remaining kinematics is no longer available to move the instrument, as the tube rigidly connected with the kinematics would hereby also move. Consequently, a tube rigidly connected with the kinematics limits its possible structure and applications.

Due to the additional movable articulated coupling, the tube flange or a tube coupled to it can be moved relative to the kinematics and thus make possible, in one embodiment, greater flexibility in structuring and/or using the medical robot, in particular its kinematics.

Thus, in one embodiment, the articulated coupling has one or more additional, respectively external to the kinematics, translational degrees of freedom. In particular, the articulated coupling can have a translational degree of freedom in one direction which is at least substantially parallel to a direction of the instrument longitudinal axis of the instrument flange. Instrument longitudinal axis direction is understood in particular to mean primarily the direction of a longitudinal axis of a minimally invasive instrument coupled to the instrument flange. In one embodiment, the instrument flange is adapted for coupling an instrument in a single position so that hereby even the instrument longitudinal axis direction is determined with respect to the instrument flange.

Due to such a degree of freedom, the instrument flange, and with it the coupled minimally invasive instrument, can be moved by the complete kinematics of the medical robot, which advantageously increases its possible structure and/or application possibilities or variants.

In particular, the articulated coupling can for this purpose have a translational degree of freedom in one direction which is at least substantially perpendicular to a joint axis next to the instrument, respectively the last joint axis of the kinematics, which can in particular be formed as a rotational axis. In another embodiment, the direction of a translational degree of freedom of the articulated coupling with the joint axis next to the instrument of the kinematics, in particular an axis of rotation next to the instrument, can have an acute angle which is advantageously at least 5° and/or at most 45°.

Additionally or alternatively, the articulated coupling can have a translational degree of freedom in one direction which, at least substantially, is perpendicular to an instrument longitudinal axis of the instrument flange and/or parallel to an axis of rotation next to the instrument, respectively the last joint axis of the kinematics.

Additionally or alternatively to one or more translational degrees of freedom, the articulated coupling, in one embodiment of the present invention, has one or more additional, respectively external to the kinematics, rotational degrees of freedom. In particular, the articulated coupling can have a rotational degree of freedom about a rotational axis which is at least substantially perpendicular to a longitudinal instrument axis of the instrument flange, in particular two rotational degrees of freedom which are at least substantially perpendicular to the instrument longitudinal axis and/or to one another.

Due to such rotational degrees of freedom, in one embodiment a misalignment between the instrument longitudinal axis direction and a tube coupled to the tube flange can be compensated.

Additionally or alternatively, the articulated coupling can have a rotational degree of freedom which in particular is aligned with an instrument longitudinal direction of the instrument flange and is at least substantially parallel to it. In particular, in one embodiment a reorientation of the kinematics about the instrument longitudinal axis can be made possible hereby without the tube exerting an inadmissible shear load on the patient.

Additionally or alternatively, the articulated coupling can have a rotary degree of freedom about a rotational axis which is at least substantially perpendicular or parallel to an axis of rotation next to the instrument, respectively the last joint axis, in particular a rotational axis of the kinematics and/or perpendicular or parallel to a direction of a translational degree of freedom of the articulated coupling.

For representing a translational degree of freedom in addition to the kinematics, the articulated coupling has at least one embodiment a joint which is translational and external to the kinematics—or an additional joint—which is not a part of the kinematics or is not positioned between the instrument flange and the base. Such a translational joint can in particular have—or in particular be—a linear guide.

In particular, for representing a rotational degree of freedom additional to the kinematics, the articulated coupling has, in one embodiment, at least one joint which is rotational and external to the kinematics—or an additional joint—which is not part of the kinematics or is not located between the instrument flange and the base. Such a rotary joint can in particular have a single rotational degree of freedom or two or three rotational degrees of freedom, and can in particular be a universal joint. In one embodiment, a translational degree of freedom can also be or be represented by two or more rotary joints of the articulated coupling, which have substantially parallel axes of rotation in one embodiment.

One joint of the articulated coupling can, in one embodiment, be mounted in plain or anti-friction bearings in order to reduce friction and/or increase the precision and/or the maximum bearing load.

In one embodiment, one or more joints of the articulated coupling can be or are actuated in particular hydraulically, pneumatically or electrically, in particular using electric motors or electromagnetically, i.e. they have an actuator, for example an electric motor, for moving or adjusting the joint. In one embodiment, the tube flange can hereby be actively moved relative to the kinematics.

In one embodiment, an end effector can be actuated to couple to the instrument flange by means of a motion of the instrument shaft relative to a coupling of the instrument. Then, in one embodiment, the end effector of a coupled minimally invasive instrument can be actuated due to the actuated articulated coupling or its motion relative to the kinematics, respectively its instrument flange.

Additionally or alternatively, in one embodiment one or more joints of the articulated coupling are passive, i.e. they have no actuator for moving the joint. A passive flexibility can be represented hereby in one embodiment. In one embodiment, one or more passive joints are elastically restrained, in particular pneumatically and/or hydraulically or mechanically, possibly by springs. A mechanically restrained joint is understood to mean one which, when an external load is removed, automatically seeks or returns to its unloaded zero position.

In one embodiment, one or more active and/or passive, in particular elastically restrained joints, are lockable, in particular in order to fix a joint setting. A joint can in one embodiment be frictionally and/or positively lockable, in particular by a detachable clamp connection and/or a preferably pre-loaded snap-on connection with one or more movable elements which, preferably with pre-loading, engage into recesses. An element can, in one embodiment, be a bolt or a sphere which is pressed into a recess by a spring means in order to lock a joint. In one other embodiment, an element can have an elastic latching hook which grips an undercut so as to lock a joint.

In one embodiment, at least one joint is detachably and/or repeatedly or repetitively lockable. In one embodiment, it can automatically lock if a single predefined or one of many predefined joint positions are reached, for example in that a pre-loaded element upon reaching the joint position engages into a recess that is then aligned with it. Generally, an active or passive joint can be locked, in one embodiment, in exactly one or more discrete or infinitely many joint positions.

In one embodiment, at least one joint of the articulated coupling is manually lockable and/or its locking is manually releasable. Additionally or alternatively, at least one lockable joint of the articulated coupling can have actuated locking and/or un-locking, in particular an electric motor or electromagnetic latching. Thus, in one embodiment, an electromagnet or electric motor can guide an element into and/or out of a recess in order to lock a joint or release the lock, i.e. unlock it.

In one embodiment of the present invention, the articulated coupling is connected, in particular detachably, with an end link of the kinematics or the instrument flange. In one embodiment, the entire kinematics can hereby be used for prepositioning the articulated coupling.

In another embodiment, the articulated coupling is connected, in particular detachably, with a starting link of the kinematics or of the base. In one embodiment, the entire kinematics is hereby available for positioning the instrument flange.

Likewise, the articulated coupling can be connected, in particular detachably, with an intermediate link of the kinematics, which connects two joints of the kinematics with one another. One part of the kinematics is available for general pre-positioning of the instrument and tube flange, another part of the kinematics for positioning or moving the instrument and tube flange relative to one another.

In one embodiment, the tube flange is weight-compensated, substantially at least, by the articulated coupling. Here in particular it is understood that the weight force of the tube flange and the articulated coupling connected with it acts, not completely at least, on the tube flange and is thus disadvantageously supported by the patient through the coupled tube.

In one embodiment, the tube flange can be passively weight-compensated through the articulated coupling, in particular by suitable elastically restrained passive joints. Additionally or alternatively, the tube flange can be actively weight-compensated through the articulated coupling, in particular through correspondingly actuated active joints.

In one embodiment, one or more joints of the articulated coupling have measuring means, in particular a sensor, for determining a force which operates on or in the joint, where presently for compact representation even an anti-parallel pair of forces, i.e. a torque, is generally called a force, the measuring means can thus also determine a torque. Through the determination of forces in the articulated coupling, an excessive load on the articulated coupling and/or the attached medical robot kinematics and/or of the tube and thus the patient can be determined. The medical robot, in particular its articulated coupling, can react thereto suitably, for example by yielding. In particular, the aforementioned active weight compensation can be implemented using of such measurement means.

According to a further aspect of the present invention, which can be combined with the aforementioned aspect, an instrument longitudinal direction of the instrument flange intersects an axis next to the instrument or the last joint axis of the kinematics formed as an axis of rotation, at an angle. This angle amounts in one embodiment to at least +5° or −5°, in particular at least +15° or −15°, and/or at most +85° or 85°, in particular at most +75° or −75°. The instrument longitudinal axis direction and the joint axis can, in one embodiment, intersect in a trocar point of the tube flange.

In one embodiment, a rotational degree of freedom of the minimally invasive instrument about the trocar point can be represented substantially alone by the last joint axis of the kinematics, another rotational axis by a planar movement of the kinematics. Additionally or alternatively, a more compact kinematics, in particular the instrument mounting, can be provided through this aspect, in particular compared with the construction of EP 1 015 068 A1.

In one embodiment, the kinematics and/or the articulated coupling has a so-called chain structure, wherein each joint is directly connected with exactly one preceding joint and at most one subsequent joint. Kinematics and articulated coupling together can form a tree structure in one embodiment. In particular, the articulated coupling can branch at a starting, intermediate or end link of the kinematics. The control of the medical robot can hereby be improved.

In one embodiment, the articulated coupling has exactly one, exactly two or exactly three joints: using a single joint in one embodiment with very compact construction, a well defined, simple mobility, in particular yielding in a predetermined direction, can be realized. Two or three joints can convey, also with compact construction, a sufficient movement or movement possibility of the tube flange relative to the kinematics.

In one embodiment, the articulated coupling has a sterile cover, which covers the articulated coupling completely or in part. This can be expedient, in particular in an actuated articulated coupling, where the actuators or joint drives thereof cannot or can only conditionally be sterilized. The sterile covering can, in one embodiment, also cover the kinematics wholly or in part.

Additional advantages and features are revealed in the sub-claims and the exemplary embodiments. Shown for this purpose, partly in schematic form:

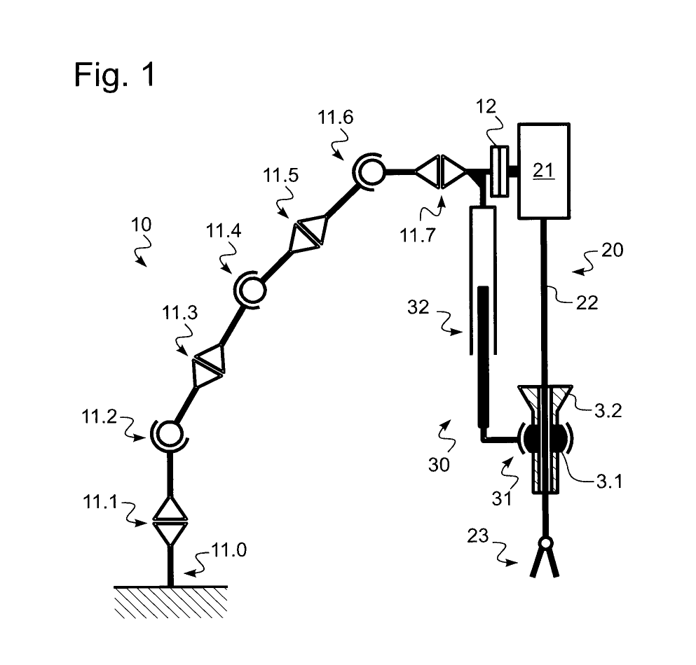

FIG. 1: a medical robot device with a medical robot according to one embodiment of the present invention;

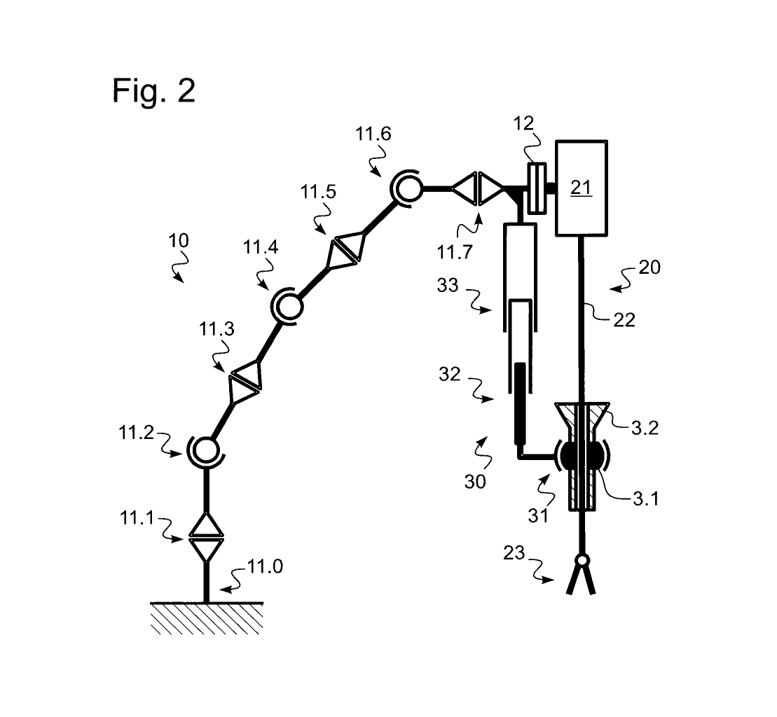

FIG. 2: a medical robot device with a medical robot according to another embodiment of the present invention;

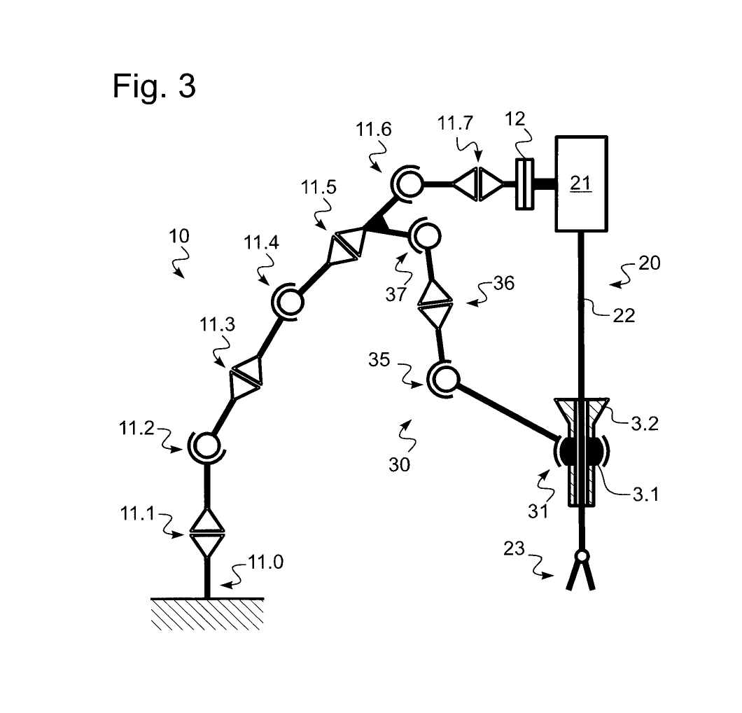

FIG. 3: a medical robot device with a medical robot according to an additional embodiment of the present invention; and

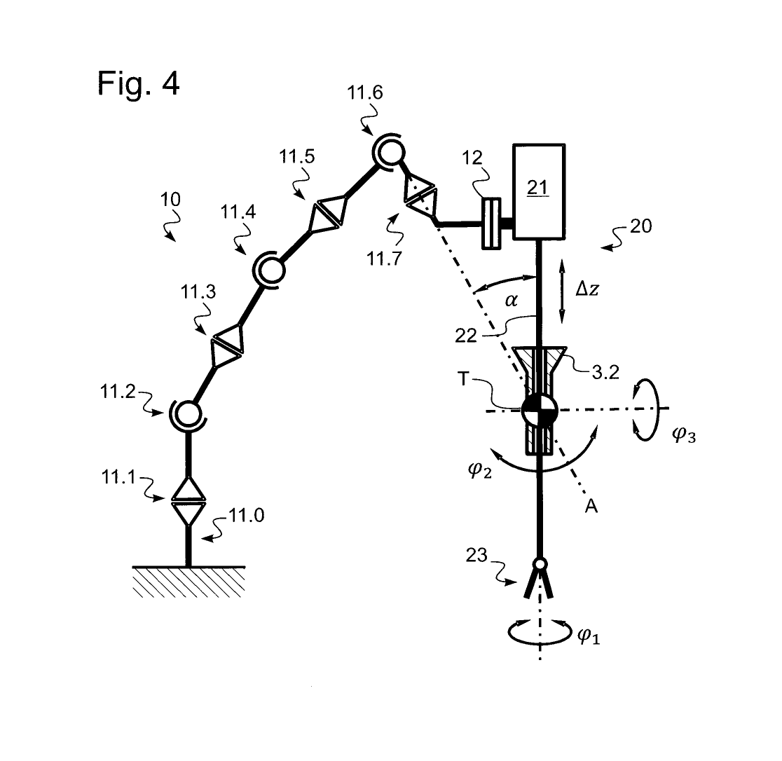

FIG. 4: a medical robot device with a medical robot according to an embodiment of the present invention.

FIG. 1 shows a medical robot device with a medical robot 10 according to one embodiment of the present invention.

Medical robot 10 has an inertial base, or one fixed to its surroundings 11.0, and an instrument flange 12, to which a minimally invasive instrument 20 is coupled. The minimally invasive instrument 20 has an instrument shaft 22 with an end effector 23, which is provided for insertion into a patient (not shown) and in the exemplary embodiment is shown for example as shears.

In the exemplary embodiment, the end effector 23 has two intra-corporal degrees of freedom for example (swivel movement of the blades of the shears) relative to the instrument shaft 22, i.e. rotational degrees of freedom about axes of rotation separated from a trocar point T (c.f. FIG. 4). For actuating these degrees of freedom, a drive 21 is positioned on the instrument shaft 22 and coupled mechanically with the end effector 23, for example through tension cables and/or push-pull rods and/or rotary shafts.

In the exemplary embodiment, the instrument 20 is coupled with a drive 21 at the instrument flange 12; in a variation that is not shown it can also, with its instrument shaft 22, in particular a housing distant from the end effector for the drive 21, be coupled to the instrument flange 12.

The instrument flange 12 is movably connected with the base 11.0 through actuated kinematics of the medical robot 10. The kinematics has seven rotary joints 11.1-11.7, each of which is actuated or movable by an electric motor. All subsequent rotational or joint axes of the kinematics are respectively perpendicular to one another.

In order to insert the end effector and a portion of the instrument shaft 22 into the patient, a tube 3.2 is provided.

The medical robot 10 is adapted to move the instrument shaft 22 about an invariant trocar point T, respectively fixed to its surroundings (c.f. FIG. 4), which is positioned inside the tube 3.2. In particular, the medical robot 10, by moving the joints 11.1-11.7 of its kinematics, moves the minimally invasive instrument 20 in the direction of an instrument longitudinal axis (vertical in FIG. 1), which is aligned with an axis direction of the tube 3.2 and includes the trocar point T (c.f. FIG. 4), in particular in order to insert it (deeper) into the patient or (at least partially) to withdraw it from the patient. In addition, the medical robot 10 can, by moving the joints 11.1-11.7 of its kinematics, rotate the minimally invasive instrument about the instrument longitudinal axis (c.f. φ₁ in FIG. 4) and about two additional rotational axes (c.f. φ₂, φ₃ in FIG. 4), which are perpendicular to one another and to the instrument longitudinal axis, and intersect at the trocar point T.

In this manner, the rotary joints 11.1-11.7 provide one translational and three rotational degrees of freedom of the end effector 23 about the trocar point T. In addition to these are the two aforementioned intra-corporal degrees of freedom, i.e. the swiveling of the shear blades, which are extra-corporally actuated by the drive 21.

The medical robot 10 has a tube flange 3.1 for detachable coupling of the tube 3.2. This tube flange 3.1 is connected with the kinematics through an additional movable articulated coupling 30.

In the exemplary embodiment of FIG. 1, the articulated coupling 30 has a passive translational joint in the form of a linear guide 32. In addition, the articulated coupling has an additional passive rotary joint with three orthogonal rotational degrees of freedom in the form of a universal joint 31. An inner ring of the universal joint 31 forms the tube flange 3.1 to which the tube 3.2 is coupled, for example by friction.

The articulated coupling 30 thereby has an additional translational degree of freedom external to the kinematics in a direction parallel to an instrument longitudinal axis of the instrument flange 12 (vertical in FIG. 1) and perpendicular to a rotational axis next to the instrument, respectively the last joint 11.7 of the kinematics. In a variation that is not shown, one or more translational degrees of freedom can also be represented by two or more rotary joints, as this is explained hereafter with reference to rotary joints 35, 37 of FIG. 3.

In addition to a translational degree of freedom, the articulated coupling has additional rotational degrees of freedom through the universal joint 31, namely two rotational degrees of freedom about axes of rotation which are perpendicular to the instrument longitudinal axis and to one another, as well as a rotational degree of freedom about an axis of rotation which is aligned with the instrument longitudinal axis of the instrument flange 12. This axis of rotation is perpendicular to joint 11.7—the axis of rotation next to the instrument—and parallel to a direction of the translational degree of freedom of the articulated coupling, while the other two axes of rotation are perpendicular or parallel to the axis of rotation of joint 11.7 and perpendicular to this translational degree of freedom. In one variation, instead of a universal joint 31 a rotary joint with only one rotational degree of freedom about the longitudinal axis of the instrument shaft 22 is provided, in particular to make possible re-orientation of the robot 10 without changing the position and orientation of the end effector 23. In particular, for tolerance compensation, the rotary joint can have one or two additional rotational degrees of freedom, and can in particular be constructed as universal joint 31 as previously described.

Due to this movable articulated coupling 30, the complete kinematics 11.1-11.7 of the medical robot 10 can be used for moving the instrument 20. Thus for example the redundant kinematics can be moved vertically by opposite rotation of the joints 11.2, 11.4 and 11.6 of the end effector in FIG. 1, in particular with joint 11.7 not moving. The tube flange 3.1 with coupled tube 3.2 thus yields passively. Likewise, the redundant kinematics can be re-oriented, in particular about the instrument longitudinal axis direction. Through the passive universal joint 31, the tube 3.2 in the patient is then advantageously slightly impinged.

The passive joints 31, 32 are elastically restrained, in the exemplary embodiment mechanically by constant-force springs (not shown). The tube flange 3.1 is passively weight compensated thereby.

At least the linear guide 32 is frictionally and/or positively lockable in one or more, in particular infinitely many joint positions, for example by a releasable clamp connection or a pre-loaded snap lock (not shown). Locking can occur and/or be released manually and/or by an electric motor or electromagnetic latching.

The articulated coupling 30 of FIG. 1 is connected detachably with an end link of the kinematics or the instrument flange 12. In one embodiment, the entire kinematics can hereby be used for pre-positioning the articulated coupling.

FIG. 2 shows a medical robot device with a medical robot according to an additional embodiment of the present invention. Elements identical with the other embodiments are designated with identical reference symbols, so that their description can be referred to and hereafter only the differences need to be considered.

The articulated coupling 30 of the embodiment of FIG. 2 has an active translational joint in the form of an actuated telescoping cylinder 33 between the passive linear guide 32 and the end link 12 of the kinematics. The articulated coupling can thereby (with the telescoping cylinder retracted) be stowed more compactly. In addition or alternatively, an active weight compensation of the articulated coupling or of the tube flange 3.1 and the tube 3.2 coupled thereto can be provided by the telescoping cylinder 33. The passive linear guide 32 of FIG. 2 respectively 3 and/or the actuated telescoping cylinder 33 can in each case be constructed in one single or many stages, wherein one stage can have two parts moving inside one another. Thus for example a two-stage linear guide or a two-stage telescopic tube can have three concentric shells, at least partially movable within one another. In addition or alternatively to parts movable within one another, a linear guide or a telescoping cylinder, can also have a shear drive or several consecutive rotary joints, as will be explained hereafter with reference to rotary joints 35, 37 of FIG. 3. In an alternative embodiment, the articulated coupling has only one actuated telescoping cylinder, in particular in three parts, in FIG. 2 for example 32+33. In this respect FIG. 2 represents at the same time an embodiment with a passive linear guide 32 and an actuated telescoping cylinder 33 and alternatively also an embodiment with an actuated, three-part or two-stage telescoping cylinder 32+33 in one figure.

FIG. 3 shows a medical robot device with a medical robot according to an additional embodiment of the present invention. Elements identical with the other embodiments are designated with identical reference symbols, so that their description can be referred to and hereafter only the differences will be discussed.

The articulated coupling 30 of the embodiment of FIG. 3 is detachably mounted to an intermediate link of the kinematics between its joints 11.5 and 11.6. It has no translational joints. Instead, the previously mentioned translational degree of freedom parallel to the instrument longitudinal axis (vertical in FIG. 3) is in particular shown by two parallel rotary joints 35, 37 of the articulated coupling. These can be kinematically coupled in a further development in order to actuate the translational degree of freedom with a single drive. In particular, in order for the last rotary joint 11.7 of the kinematics to be usable for moving the instrument 20, an additional rotary joint 36, the axis of rotation whereof is perpendicular to the axes of rotation of the rotary joints 35, 37, is positioned between the parallel rotary joints 35, 37.

The rotary joints 35-37 of the articulated coupling are actuated by electric motors (not shown). The tube flange 3.1 can hereby firstly be actively weight-compensated by the articulated coupling, as explained earlier with reference to FIG. 2. In addition, the tube flange 3.1 can be actively moved relative to the kinematics 11.1-11.7. This can be used in order to actuate the end effector 23: consider for example the two shear blades coupled with the tube 3.2; a movement of the tube 3.2 by means of the articulated coupling 20 relative to the instrument flange 12 causes a movement of the shear blades relative to the instrument shaft 22.

FIG. 4 shows a medical robot device with a medical robot according to another embodiment of the present invention. Elements identical with the other embodiments are designated with identical reference symbols, so that their description can be referred to and hereafter only the differences will be discussed.

In this embodiment, the tube 3.2 can be free or coupled to a tube flange (not shown in FIG. 4). An instrument longitudinal axis direction of the instrument flange 12, or that of the coupled instruments 20, intersects a joint axis A of the rotary joint next to the instrument or last rotary joint 11.7 at the trocar point T of the tube 3.2 at an angle α, which amounts to about 30° in the exemplary embodiment.

The rotational degree of freedom φ₃ of the minimally invasive instrument 20 about the trocar point can hereby be substantially represented by the last joint axis 11.7 of the kinematics, the other rotational degree of freedom φ₂ by a planar movement of the kinematics 11.1-11.7. In addition, this inclination provides a more compact kinematics, in particular instrument mounting.

In the exemplary embodiments, the kinematics 11.1-11.7 and the articulated coupling 3.1-3.4 both have a chain structure, kinematics and articulated coupling consequently form a tree structure together.

### REFERENCE SYMBOL LIST

10 medical robot

11.0 base

11.1-11.7 actuated rotary joint (kinematics)

12 instrument flange, end link

20 minimally invasive instrument

21 drive

22 instrument shaft

23 shears (end effector)

3.1 tube flange

3.2 tube

30 articulated coupling

31 universal joint

32 linear guide (passive translational joint)

33 telescoping cylinder (active translational joint)

35-37 active rotary joint (articulated coupling)

T trocar point

A joint axis

1. Medizinroboter (10) mit:
einer Basis (11.0);
einem Instrumenten-Flansch (12) zum Ankoppeln eines minimalinvasiven Instruments (20), der durch eine aktuierte Kinematik (11.1-11.7) verstellbar mit der Basis verbunden ist; and
einem Tubus-Flansch (3.1) zum Ankoppeln eines Tubus (3.2), dadurch gekennzeichnet, dass der Tubus-Flansch durch eine bewegliche Gelenkanordnung (30) mit der Kinematik verbunden ist, wobei die Gelenkanordnung wenigstens ein, insbesondere elektrisch, aktuiertes und/oder wenigstens ein elastisch gefesseltes passives und/oder wenigstens ein arretierbares Gelenk (31-37) aufweist.

2-10. (canceled)
